(19) United States
(12) Patent Application Publication
(10) Pub. No.: US 2007/0239193 A1
(43) Pub. Date: Oct. 11, 2007

(54) Stretch-resistant vaso-occlusive devices with distal anchor link

(75) Inventors: Joan Simon, Sunnyvale, CA (US); Cindy Truong, San Jose, CA (US)

Correspondence Address: ROBINS & PASTERNAK, 1731 EMBARCADERO ROAD, SUITE 230, PALO ALTO, CA, 94303, US

(73) Assignee: Boston Scientific Scimed, Inc.

(21) Appl. No.: 11/400,100

(22) Filed: Apr. 5, 2006

(51) Int. Cl. A61M 29/00

(52) U.S. Cl. 606/191; 606/200

(57) ABSTRACT

Disclosed herein are vaso-occlusive devices for forming occluding the vasculature of a patient. More particularly, disclosed herein are vaso-occlusive devices comprising a stretch-resistant member including at least one anchor link. Also disclosed are methods of making and using these devices.

### FIELD OF THE INVENTION

Devices and methods for repair of aneurysms are described. In particular, stretch-resistant vaso-occlusive devices comprising an anchor link structure that imparts high tensile strength to the device are described.

### BACKGROUND

An aneurysm is a dilation of a blood vessel that poses a risk to health from the potential for rupture, clotting, or dissecting. Rupture of an aneurysm in the brain causes stroke, and rupture of an aneurysm in the abdomen causes shock. Cerebral aneurysms are usually detected in patients as the result of a seizure or hemorrhage and can result in significant morbidity or mortality.

There are a variety of materials and devices which have been used for treatment of aneurysms, including platinum and stainless steel microcoils, polyvinyl alcohol sponges (Ivalone), and other mechanical devices. For example, vaso-occlusion devices are surgical implements or implants that are placed within the vasculature of the human body, typically via a catheter, either to block the flow of blood through a vessel making up that portion of the vasculature through the formation of an embolus or to form such an embolus within an aneurysm stemming from the vessel. One widely used vaso-occlusive device is a helical wire coil having windings that may be dimensioned to engage the walls of the vessels. (See, e.g., U.S. Pat. No. 4,994,069 to Ritchart et al.).

Coil devices including polymer coatings or attached polymeric filaments have also been described. See, e.g., U.S. Pat. Nos. 5,226,911; 5,935,145; 6,033,423; 6,280,457; 6,287,318; and 6,299,627. For instance, U.S. Pat. No. 6,280,457 describes wire vaso-occlusive coils having single or multi-filament polymer coatings. U.S. Pat. Nos. 6,287,318 and 5,935,145 describe metallic vaso-occlusive devices having a braided polymeric component attached thereto. U.S. Pat. No. 5,382,259 describes braid structures covering a primary coil structure.

In addition, coil designs including stretch-resistant members comprising thermoplastic polymeric fibers that run through the lumen of the helical vaso-occlusive coil and are secured to the coil by heat treatment have also been described. See, e.g., U.S. Pat. Nos. 5,582,619; 5,833,705; 5,853,418; 6,004,338; 6,013,084; 6,179,857; and 6,193,728.

U.S. Pat. Nos. 6,620,152; 6,425,893; 5,976,131, 5,354,295; and 5,122,136, all to Guglielmi et al., describe electrolytically detachable embolic devices. U.S. Pat. No. 6,623,493 describes vaso-occlusive member assembly with multiple detaching points. U.S. Pat. Nos. 6,589,236 and 6,409,721 describe assemblies containing an electrolytically severable joint.

There remains a need for stretch-resistant vaso-occlusive devices having higher tensile strength as well as methods of making and using such devices.

### SUMMARY OF THE INVENTION

Thus, this invention includes novel occlusive compositions as well as methods of using and making these compositions.

In one aspect, the invention relates to a vaso-occlusive device comprising a core element having a proximal end and a distal end; and a stretch-resistant member secured to at least two locations to the core element, the stretch-resistant member comprising an anchor link including an eyelet and at least one filament extending through the eyelet of the anchor link. In certain embodiments, the filament further comprises a knot therein such that the filament creates a loop. The device may also comprise a pusher wire for use in delivery. The optional pusher wire comprises a proximal and distal end and is preferably detachably connected to the vaso-occlusive device, for example, at the proximal end of the device.

The anchor link may be secured to one or more locations of the core element, for example, to the pusher wire (e.g., the distal end of the pusher wire); the distal end of the core element; and/or proximal end of the core element. The anchor link may be secured using, for example, one or more adhesives.

In any of the devices described herein, the anchor link can comprise a metal (e.g., platinum) and/or one or more polymers (e.g., suture materials). Similarly, the filament may comprise one or more metals or, alternatively, one or more polymers (e.g., suture materials).

In any of the devices described herein, the core element may define a lumen and the stretch-resistant member may extend at least partially through the lumen. In certain embodiments, the core element comprises a wire formed into a helically wound primary shape. The core element may also have a secondary shape (e.g., cloverleaf shaped, helically-shaped, figure-8 shaped, flower-shaped, vortex-shaped, ovoid, randomly shaped, and substantially spherical shapes) that self-forms upon deployment.

In any of the devices described herein, the core element can comprise a metal, for example, platinum, rhodium, gold, tungsten and/or alloys thereof. In certain embodiments, the core element comprises a nickel-titanium alloy.

Any of the devices described herein may further comprise one or more additional materials, for example, at least one bioactive material. Any of the devices described herein may further comprise a severable junction detachably which may be connected to a pusher element. The detachment junction can be positioned anywhere on the device, for example at one or both ends of the device. In certain embodiments, the severable junction(s) are, an electrolytically detachable assembly adapted to detach by imposition of a current; a mechanically detachable assembly adapted to detach by movement or pressure; a thermally detachable assembly adapted to detach by localized delivery of heat to the junction; a radiation detachable assembly adapted to detach by delivery of electromagnetic radiation to the junction or combinations thereof.

In another aspect, a method of occluding a body cavity is described, the method comprising introducing any of the devices as described herein into the body cavity. In certain embodiments, the body cavity is an aneurysm.

These and other embodiments of the subject invention will readily occur to those of skill in the art in light of the disclosure herein.

### BRIEF DESCRIPTION OF THE FIGURES

FIG. 1 is a side view depicting an exemplary anchor link as described herein.

FIG. 2 is a side view depicting another exemplary anchor link as described herein.

FIG. 3 is a side view depicting yet another exemplary anchor link as described herein.

FIG. 4 is a side view depicting yet another exemplary anchor link as described herein, in which the eyelet is integrated into the ball structure.

FIG. 5 is a side view depicting a stretch-resistant member comprising the anchor link shown in FIG. 1 and a filament looped through the anchor link and knotted.

FIG. 6 is side and cross-section view of the stretch-resistant member depicted in FIG. 5 in combination with a vaso-occlusive coil.

FIG. 7, panels A and B, are reproductions of photographs of showing top overviews of previously-described heat-treated stretch-resistant coils (FIG. 7A) and vaso-occlusive devices including stretch-resistant members having an anchor link (FIG. 7B) as described herein. The devices including stretch-resistant members as described herein (FIG. 7B) exhibit more consistent shapes (e.g., less variation in the outer diameter) as compared to stretch-resistant devices in which the stretch-resistant member has been heat treated (FIG. 7A).

### DETAILED DESCRIPTION

Stretch-resistant occlusive (e.g., embolic) compositions are described. The compositions described herein find use in vascular and neurovascular indications and are particularly useful in treating aneurysms, for example small-diameter, curved or otherwise difficult to access vasculature, for example aneurysms, such as cerebral aneurysms. Methods of making and using these vaso-occlusive devices are also aspects of this invention.

Unlike previously described stretch resistant vaso-occlusive coils, the devices described herein exhibit enhanced stretch resistance (tensile strength), in part, because one or both ends of the stretch-resistant members are not heat treated and, accordingly, retain their full tensile strength. Furthermore, the stretch-resistant members comprising an anchor link described herein have a structure that is more uniform throughout its entirety as compared to previously-described stretch-resistant designs.

Advantages of the present invention include, but are not limited to, (i) the provision of stretch-resistant vaso-occlusive devices with high tensile strength; (ii) the provision of stretch-resistant devices that result in structures having more uniform dimensions (e.g., in terms of the outer diameter remaining more consistent along its entire length); (iv) the provision of occlusive devices that can be retrieved and/or repositioned after deployment; and (v) cost-effective production of these devices.

All publications, patents and patent applications cited herein, whether above or below, are hereby incorporated by reference in their entirety.

It must be noted that, as used in this specification and the appended claims, the singular forms “a”, “an”, and “the” include plural referents unless the content clearly dictates otherwise. Thus, for example, reference to a device comprising “a stretch-resistant member” includes devices comprising of two or more stretch-resistant members.

The stretch-resistant members described herein comprise an anchor link structure that typically serves as the load bearing component of the stretch-resistant device. The anchor link structure may take a variety of forms including ball (sphere) shapes, ovoid shapes, half-spheres, half-ovals, cylinders, cones, etc. It is preferable that the anchor link define at least one eyelet (e.g., U-, O- or C-shaped structure and the like).

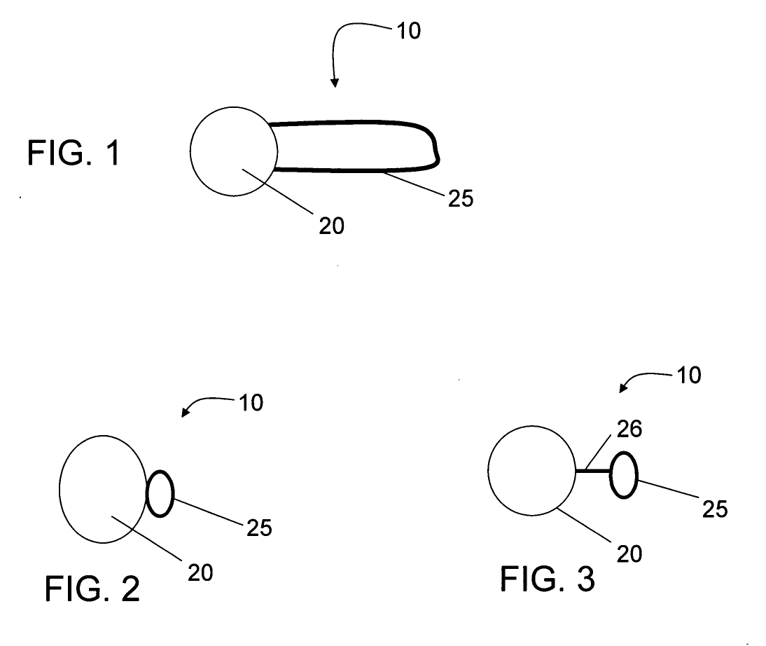

FIG. 1 shows an exemplary anchor link 10 comprising a ball-like structure 20 and a U-shaped structure 25 such that an eyelet is formed by the U-shaped structure 25.

FIG. 2 shows another exemplary anchor link 10 comprising an ball-like structure 20 and an oval structure 25 that forms an eyelet. In this embodiment, the eyelet formed by the oval structure 25 abuts the ball-like structure 20.

FIG. 3 shows yet another exemplary anchor link 10 comprising a ball-like structure 20 and another oval structure 25 that forms an eyelet. In this embodiment, the eyelet 25 includes extension 26 such that the eyelet 25 does not directly contact the ball structure 25.

FIG. 4 shows yet another exemplary anchor link 10 design in which the eyelet 25 is integrated into the ball structure 20.

The anchor link 10 may be made of any metal or polymer, including, but not limited to, the metals and polymers described below. In certain embodiments, the anchor link comprises a metal, for example, platinum, rhodium, palladium, rhenium, as well as tungsten, gold, silver, tantalum, and/or alloys thereof, including any of the metals and alloys described below. In a particularly preferred embodiment, the anchor link comprises platinum.

As noted above, the anchor link 10 may be produced as an integral structure or, alternatively, may be produced by combining two previously produced structures to form the anchor link. In certain embodiments, the ball-like structure is created by welding (e.g., microarc welding) or otherwise melting a metal or polymer into a rounded structure.

The stretch-resistant members described herein preferably include one or more filament components. The filament(s) may be attached to the anchor link in any suitable manner, for example by tying, winding, gluing, melting, etc.

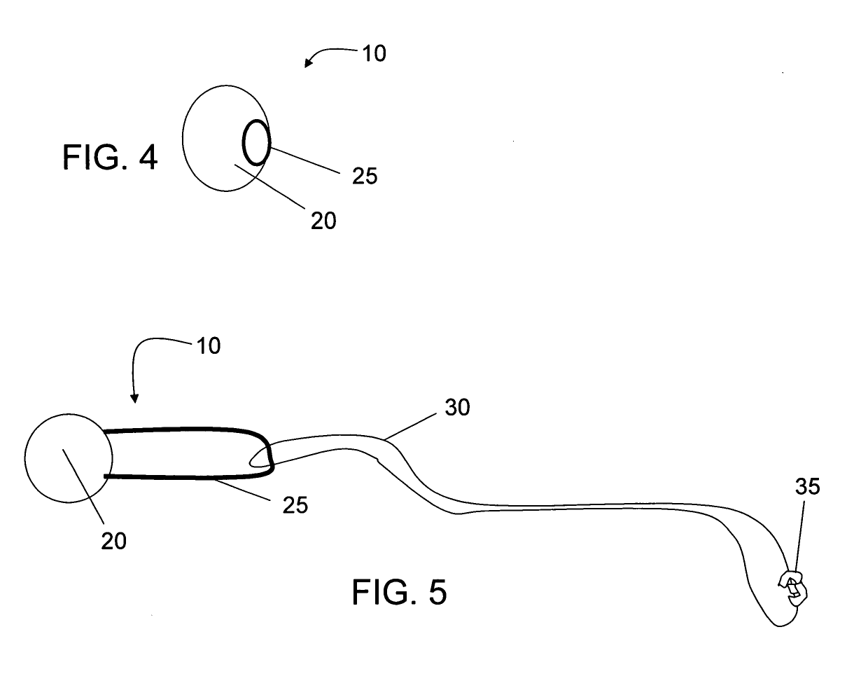

FIG. 5 shows an embodiment in which a filament 30 is extended through the eyelet 25 of the anchor link 10 and knotted 35 to form a loop that interlocks with the eyelet of the anchor link.

Filament 30 component that extends through the eyelet of the anchor link structure may be made of one or metals and/or polymers. In certain preferred embodiments, the anchor link 10 comprises a metal (e.g., platinum) and the filament component 30 comprises a polymeric filament, for example a suture material. Exemplary polymers are described below. In addition, the filament component 30 may include two or more filaments, for example constructs comprising filamentous elements assembled by one or more operations including coiling, twisting, braiding, weaving or knitting of the filamentous elements.

Non-limiting examples of polymers suitable for use in the stretch-resistant devices described herein (e.g., anchor link, filament and/or core element) include synthetic and natural polymers, such as polyurethanes (including block copolymers with soft segments containing esters, ethers and carbonates), polyethers, polyamides (including nylon polymers and their derivatives), polyimides (including both thermosetting and thermoplastic materials), acrylates (including cyanoacrylates), epoxy adhesive materials (two part or one part epoxy-amine materials), olefins (including polymers and copolymers of ethylene, propylene butadiene, styrene, and thermoplastic olefin elastomers), fluoronated polymers (including polytetrafluoroethylene), polydimethyl siloxane-based polymers, cross-linked polymers, non-cross linked polymers, Rayon, cellulose, cellulose derivatives such nitrocellulose, natural rubbers, polyesters such as lactides, glycolides, trimethylene carbonate, caprolactone polymers and their copolymers, hydroxybutyrate and polyhydroxyvalerate and their copolymers, polyether esters such as polydioxinone, anhydrides such as polymers and copolymers of sebacic acid, hexadecandioic acid and other diacids, or orthoesters may be used.

Thus, polymers used in the devices described herein (e.g., in the filament component of the stretch-resistant member) may include one or more absorbable (biodegradable) polymers and/or one or more non-absorbable polymers. The terms “absorbable” and “biodegradable” are used interchangeable to refer to any agent that, over time, is no longer identifiable at the site of application in the form it was injected, for example having been removed via degradation, metabolism, dissolving or any passive or active removal procedure. Non-limiting examples of absorbable proteins include synthetic and polysaccharide biodegradable hydrogels, collagen, elastin, fibrinogen, fibronectin, vitronectin, laminin and gelatin. Many of these materials are commercially available. Fibrin-containing compositions are commercially available, for example from Baxter. Collagen containing compositions are commercially available, for example from Cohesion Technologies, Inc., Palo Alto, Calif. Fibrinogen-containing compositions are described, for example, in U.S. Pat. Nos. 6,168,788 and 5,290,552. Mixtures, copolymers (both block and random) of these materials are also suitable.

Preferred biodegradable polymers include materials used as dissolvable suture materials, for instance polyglycolic and/or polylactic acids (PGLA) to encourage cell growth in the aneurysm after their introduction. Preferred non-biodegradable polymers include polyethylene teraphthalate (PET or Dacron), polypropylene, polytetraflouroethylene, or Nylon materials. Highly preferred are PET or PGLA.

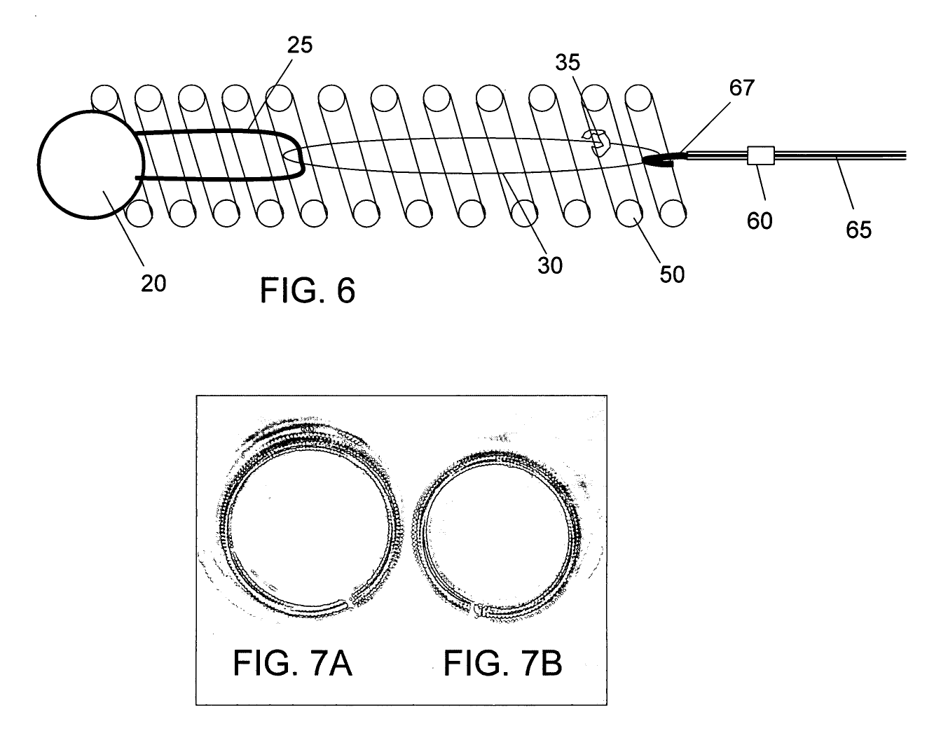

The stretch-resistant members comprising an anchor link described herein are combined with a vaso-occlusive core element so as to inhibit unwanted stretching of the vaso-occlusive core element. Typically, although not required, the anchor link is approximately the same diameter as the core element. FIG. 6 depicts a stretch-resistant member as shown in FIG. 5 (including anchor link 20, 25 and knotted 35 filament 30) in combination with a coil-shaped vaso-occlusive core element 50.

The core element may be made of a variety of materials (e.g., metal, polymer, etc.), including the polymers and metals described above. Although depicted in the Figures as a helically wound metallic coil, it will be appreciated that the drawings are for purposes of illustration only and that other embolic devices may be of a variety of shapes or configuration including, but not limited to, open and/or closed pitch helically wound coils, braids, wires, knits, woven structures, tubes (e.g., perforated or slotted tubes), injection-molded devices and the like. See, e.g., U.S. Pat. No. 6,533,801 and International Patent Publication WO 02/096273.

In a particularly preferred embodiment, the core element comprises at least one metal or alloy. Suitable metals and alloys for use in the core element, anchor link and/or filament(s) include the Platinum Group metals, especially platinum, rhodium, palladium, rhenium, as well as tungsten, gold, silver, tantalum, and alloys of these metals. In one preferred embodiment, the core element comprises platinum. The core element may also comprise of any of a wide variety of stainless steels if some sacrifice of radio-opacity may be tolerated. Very desirable materials of construction, from a mechanical point of view, are materials that maintain their shape despite being subjected to high stress.

Certain “super-elastic alloys” include nickel/titanium alloys (48-58 atomic % nickel and optionally containing modest amounts of iron); copper/zinc alloys (38-42 weight % zinc); copper/zinc alloys containing 1-10 weight % of beryllium, silicon, tin, aluminum, or gallium; or nickel/aluminum alloys (36-38 atomic % aluminum) may also be used to make the core element, anchor link and/or in the filaments of the stretch-resistant devices described herein. Particularly preferred for the core element are the alloys described in U.S. Pat. Nos. 3,174,851; 3,351,463; and 3,753,700. Especially preferred is the titanium/nickel alloy known as “nitinol.” These are very sturdy alloys that will tolerate significant flexing without deformation even when used as a very small diameter wire. If a super-elastic alloy such as nitinol is used in any component of the device, the diameter of the wire may be significantly smaller than that used when the relatively more ductile platinum or platinum/tungsten alloy is used as the material of construction. These metals have significant radio-opacity and in their alloys may be tailored to accomplish an appropriate blend of flexibility and stiffness. They are also largely biologically inert.

The core element may have a primary and secondary (relaxed configuration). In certain embodiments, the core element changes shape upon deployment, for example change from a constrained linear form to a relaxed, three-dimensional (secondary) configuration. See, also, U.S. Pat. No. 6,280,457 and documents cited above for methods of making vaso-occlusive coils having a linear helical shape and/or a different three-dimensional (secondary) configuration.

Thus, it is further within the scope of this invention that the vaso-occlusive device as a whole or elements thereof comprising secondary shapes or structures that differ from the linear coil shapes depicted in the Figures, for examples, spheres, ellipses, spirals, ovals, figure-8 shapes, etc. The devices described herein may be self-forming in that they assume the secondary configuration upon deployment into an aneurysm. Alternatively, the devices may assume their secondary configurations under certain conditions (e.g., change in temperature, application of energy, etc.).

In a preferred embodiment, the core element comprises a metal wire wound into a primary helical shape. The core element may be, but is not necessarily, subjected to a heating step to set the wire into the primary shape. The diameter of the wire typically making up the coils is often in a range of 0.0005 and 0.050 inches, preferably between about 0.001 and about 0.004 inches in diameter.

FIG. 6 also shows is a detachment junction 60 and pusher wire 65. Detachment junction 60 is preferably electrolytically detachable, but may also be adapted to be mechanically detachable (upon movement or pressure) and/or detached upon the application of heat (thermally detachable), the application of radiation, and/or the application of electromagnetic radiation. In a preferred embodiments, stretch-resistant vaso-occlusive devices as described herein are conveniently detached from the deployment mechanism (e.g., pusher wire) by the application of electrical energy, which dissolves a suitable substrate at the selected detachment junction. Methods of connecting a core element to a pusher wire having an electrolytically detachable junction are well known and described for example in U.S. Pat. Nos. 6,620,152; 6,425,893; 5,976,131, 5,354,295; and 5,122,136.

The stretch-resistant member may be secured to the core element in any fashion, including, but not limited to, melting, by adhesives (e.g., EVA), tying, winding and the like. The stretch-resistant member may be attached to the core element at one or more locations. In certain embodiments, one or both ends of the stretch-resistant member are attached to or near one or both ends of the core element.

For instance, as shown in FIG. 6, the ball component 20 of the anchor link is fixed attached (e.g., using one or more adhesives) to the distal end of the coil 50. In addition, FIG. 6 depicts an embodiment in which the filament component 30 is attached at the proximal end of the core element 50 via a hook 67 on the end of the pusher wire 65. The filament component 30 can be attached by any suitable means (e.g., gluing, tying, melting, soldering, etc.) at one or more locations of the device, so long as the attachment point(s) is(are) distal to the detachment junction 60.

In certain preferred embodiments, the anchor link and/or filament component are attached to the ends of the core element without the need for heat treatment, for example using one or more adhesives. By eliminating high temperature treatments to secure the stretch-resistant member, the tensile strength of the stretch-resistant member is enhanced. Accordingly, more force (pushing or pulling) can be applied by the physician during positioning of the device. In addition, the non-heat treated stretch-resistant devices described herein have dimensions (e.g., outer diameter (O.D)) that are more uniform (consistent throughout their length) as compared to devices in which the stretch-resistant members are heat treated (see, FIG. 7A showing heat-treated device in which the O.D. varies along the length of the device as compared to FIG. 7B showing a device as described herein in which the stretch-resistant member is not secured to the device by heat treatment).

The stretch-resistant member may be assembled in its entirety (e.g., threading the filament through the eyelet and knotting the filament prior to combining with the core element) and then combined with the core element by any suitable means, for example by securing the anchor link to the distal end of the core element and securing the filament component to the coil distal to the detachment junction. Alternatively, individual components of the stretch-resistant member may be combined with the core element before they are assembled into the stretch-resistant member. For example, an anchor link may be secured to the core element and, subsequently, the filament component may be combined with the anchor link (e.g., by threading the filament through the eyelet of the anchor link). The filament can be extended through as much of the lumen of the core element as desired and/or knotted.

Furthermore, the stretch-resistant member (or components thereof) may be combined with the core element before or after the core element is shaped into a primary and/or secondary configuration. For example, the core element may be formed into its primary configuration, one or more components of the stretch-resistant member inserted through at least part of the lumen of the primary configuration and secured to the primary configuration as desired. Alternatively, the primary configuration can be shaped into its secondary form and heat treated so that it will return to the secondary form when relaxed (deployed). One or more components of the stretch-resistant member may then be secured to the core element as desired. Whatever combination strategy is employed, the stretch-resistant member does not substantially affect the shape of the core element when the core element assumes the relaxed (secondary) configuration.

It will also be apparent that when the core element is not stretched, the stretch-resisting member would be loose, i.e., normally longer than the length (e.g., lumen) of the core element. This slack allows the device to pass through the catheter and return to its secondary form. In addition, the slack in the stretch-resistant member provides a cue to the physician about the state of the device when the device is being positioned (pulling or retracting), e.g., when there is no more slack, the device will be stretched upon further movement.

One or more of the components of the devices described herein (e.g., stretch-resistant member, core element) may also comprise additional components, such as co-solvents, plasticizers, radio-opaque materials (e.g., metals such as tantalum, gold or platinum), coalescing solvents, bioactive agents, antimicrobial agents, antithrombogenic agents, antibiotics, pigments, radiopacifiers and/or ion conductors which may be coated using any suitable method or may be incorporated into the element(s) during production.

In addition, lubricious materials (e.g., hydrophilic) materials may be used to coat one or more members of the device to help facilitate delivery. Cyanoacrylate resins (particularly n-butylcyanoacrylate), particular embolization materials such as microparticles of polyvinyl alcohol foam may also be introduced into the intended site after the inventive devices are in place. Furthermore, previously described fibrous braided and woven components (U.S. Pat. No. 5,522,822) may also be included.

One or more bioactive materials may also be included. See, e.g., co-owned U.S. Pat. No. 6,585,754 and WO 02/051460. The term “bioactive” refers to any agent that exhibits effects in vivo, for example a thrombotic agent, an anti-thrombotic agent (e.g., a water-soluble agent that inhibits thrombosis for a limited time period, described above), a therapeutic agent (e.g., chemotherapeutic agent) or the like. Non-limiting examples of bioactive materials include cytokines; extracellular matrix molecules (e.g., collagen); trace metals (e.g., copper); and other molecules that stabilize thrombus formation or inhibit clot lysis (e.g., proteins or functional fragments of proteins, including but not limited to Factor XIII, α-antiplasmin, plasminogen activator inhibitor-1 (PAI-1) or the like). Non-limiting examples of cytokines which may be used alone or in combination in the practice of the present invention include, basic fibroblast growth factor (bFGF), platelet derived growth factor (PDGF), vascular endothelial growth factor (VEGF), transforming growth factor beta (TGF-β) and the like. Cytokines, extracellular matrix molecules and thrombus stabilizing molecules (e.g., Factor XIII, PAI-1, etc.) are commercially available from several vendors such as, for example, Genzyme (Framingham, Mass.), Genentech (South San Francisco, Calif.), Amgen (Thousand Oaks, Calif.), R&D Systems and Immunex (Seattle, Wash.). Additionally, bioactive polypeptides can be synthesized recombinantly as the sequences of many of these molecules are also available, for example, from the GenBank database. Thus, it is intended that the invention include use of DNA or RNA encoding any of the bioactive molecules. Cells (e.g., fibroblasts, stem cells, etc.) can also be included. Such cells may be genetically modified. Furthermore, it is intended, although not always explicitly stated, that molecules having similar biological activity as wild-type or purified cytokines, extracellular matrix molecules and thrombus-stabilizing proteins (e.g., recombinantly produced or mutants thereof) and nucleic acid encoding these molecules are intended to be used within the spirit and scope of the invention. Further, the amount and concentration of liquid embolic and/or other bioactive materials useful in the practice of the invention can be readily determined by a skilled operator and it will be understood that any combination of materials, concentration or dosage can be used, so long as it is not harmful to the subject.

The devices described herein are often introduced into a selected site using the procedure outlined below. This procedure may be used in treating a variety of maladies. For instance in the treatment of an aneurysm, the aneurysm itself will be filled (partially or fully) with the compositions described herein.

Conventional catheter insertion and navigational techniques involving guidewires or flow-directed devices may be used to access the site with a catheter. The mechanism will be such as to be capable of being advanced entirely through the catheter to place vaso-occlusive device at the target site but yet with a sufficient portion of the distal end of the delivery mechanism protruding from the distal end of the catheter to enable detachment of the implantable vaso-occlusive device. For use in peripheral or neural surgeries, the delivery mechanism will normally be about 100-200 cm in length, more normally 130-180 cm in length. The diameter of the delivery mechanism is usually in the range of 0.25 to about 0.90 mm. Briefly, occlusive devices (and/or additional components) described herein are typically loaded into a carrier for introduction into the delivery catheter and introduced to the chosen site using the procedure outlined below. This procedure may be used in treating a variety of maladies. For instance, in treatment of an aneurysm, the aneurysm itself may be filled with the embolics (e.g. vaso-occlusive members and/or liquid embolics and bioactive materials) which cause formation of an emboli and, at some later time, is at least partially replaced by neovascularized collagenous material formed around the implanted vaso-occlusive devices.

A selected site is reached through the vascular system using a collection of specifically chosen catheters and/or guide wires. It is clear that should the site be in a remote site, e.g., in the brain, methods of reaching this site are somewhat limited. One widely accepted procedure is found in U.S. Pat. No. 4,994,069 to Ritchart, et al. It utilizes a fine endovascular catheter such as is found in U.S. Pat. No. 4,739,768, to Engelson. First of all, a large catheter is introduced through an entry site in the vasculature. Typically, this would be through a femoral artery in the groin. Other entry sites sometimes chosen are found in the neck and are in general well known by physicians who practice this type of medicine. Once the introducer is in place, a guiding catheter is then used to provide a safe passageway from the entry site to a region near the site to be treated. For instance, in treating a site in the human brain, a guiding catheter would be chosen which would extend from the entry site at the femoral artery, up through the large arteries extending to the heart, around the heart through the aortic arch, and downstream through one of the arteries extending from the upper side of the aorta. A guidewire and neurovascular catheter such as that described in the Engelson patent are then placed through the guiding catheter. Once the distal end of the catheter is positioned at the site, often by locating its distal end through the use of radiopaque marker material and fluoroscopy, the catheter is cleared. For instance, if a guidewire has been used to position the catheter, it is withdrawn from the catheter and then the assembly, for example including the absorbable vaso-occlusive device at the distal end, is advanced through the catheter.

Once the selected site has been reached, the vaso-occlusive device is extruded, for example by loading onto a pusher wire. Preferably, the vaso-occlusive device is loaded onto the pusher wire via an electrolytically cleavable junction (e.g., a GDC-type junction that can be severed by application of heat, electrolysis, electrodynamic activation or other means). Additionally, the vaso-occlusive device can be designed to include multiple detachment points, as described in co-owned U.S. Pat. Nos. 6,623,493 and 6,533,801 and International Patent publication WO 02/45596. They are held in place by gravity, shape, size, volume, magnetic field or combinations thereof.

It will also be apparent that the operator can remove or reposition (distally or proximally) the device. For instance, the operator may choose to insert a device as described herein, before detachment, move the pusher wire to place the device in the desired location.

Modifications of the procedure and vaso-occlusive devices described above, and the methods of using them in keeping with this invention will be apparent to those having skill in this mechanical and surgical art. These variations are intended to be within the scope of the claims that follow.

What is claimed is:

1. A vaso-occlusive device comprising
a core element having a proximal end and a distal end; and
a stretch-resistant member secured to at least two locations to the core element, the stretch-resistant member comprising an anchor link including an eyelet and at least one filament extending through the eyelet of the anchor link.

2. The vaso-occlusive device of claim 1, wherein the filament further comprises a knot therein such that the filament creates a loop.

3. The vaso-occlusive device of claim 1, wherein the anchor link is secured to the distal end of the core element using one or more adhesives.

4. The vaso-occlusive device of claim 1, wherein the filament is secured to the proximal end of the core element using one or more adhesives.

5. The vaso-occlusive device of claim 4, further comprising a detachable pusher wire and wherein the filament is secured to the distal end of the pusher wire.

6. The vaso-occlusive device of claim 1, wherein the anchor link comprises a metal.

7. The vaso-occlusive device of claim 6, wherein the metal comprises platinum.

8. The vaso-occlusive device of claim 1, wherein the anchor link comprises a polymer.

9. The vaso-occlusive device of claim 1, wherein the filament comprises one or more polymers.

10. The vaso-occlusive device of claim 9, wherein the polymer comprises a suture material.

11. The vaso-occlusive device of claim 1, wherein the core element defines a lumen and the stretch-resistant member extends at least partially through the lumen.

12. The vaso-occlusive device of claim 1, wherein the core element comprises a wire formed into a helically wound primary shape.

13. The vaso-occlusive device of claim 1, where the core element has a secondary shape that self-forms upon deployment.

14. The vaso-occlusive device of claim 12, where the secondary shape is selected from the group consisting of cloverleaf shaped, helically-shaped, figure-8 shaped, flower-shaped, vortex-shaped, ovoid, randomly shaped, and substantially spherical.

15. The vaso-occlusive device of claim 1, wherein the core element comprises a metal.

16. The vaso-occlusive device of claim 15, wherein the metal is selected from the group consisting of platinum, rhodium, gold, tungsten and alloys thereof.

17. The vaso-occlusive device of claim 16, wherein the metal comprises a nickel-titanium alloy.

18. The vaso-occlusive device of claim 1, further comprising a detachment junction.

19. The vaso-occlusive device of claim 18, wherein the detachment junction is electrolytically detachable.

20. A method of at least partially occluding an aneurysm, the method comprising the steps of introducing a vaso-occlusive assembly according to claim 1 into the aneurysm and detaching the core element from the detachment junction, thereby deploying the core element into the aneurysm.
